(19) United States
(12) Patent Application Publication
(10) Pub. No.: US 2019/0221286 A1
(43) Pub. Date: Jul. 18, 2019

(54) Method of Threshold Estimation in Digital PCR

(71) Applicant: Oslo universitetssykehus HF, Oslo (NO)

(72) Inventors: Guro E. Lind, Oslo (NO); Marine Jeanmougin, Oslo (NO); Heidi D. Pharo, Oslo (NO); Kim Andresen, Nesbru (NO); Ragnhild Lothe, Oslo (NO)

(21) Appl. No.: 16/245,391

(22) Filed: Jan. 11, 2019

(30) Foreign Application Priority Data
Jan. 12, 2018 (GB) 1800551.2

(51) Int. Cl. G16B 25/20; C12Q 1/686; G16B 25/10

(52) U.S. Cl. CPC G16B 25/20; G16B 25/10; C12Q 1/686

(57) ABSTRACT

A method of estimating thresholds for droplet digital polymerase chain reaction (ddPCR) experiments is described. The method comprises receiving amplitude values for multiple ddPCR experiments and, for each experiment, determining if the distribution of amplitude values is multimodal or unimodal. In the case of unimodality where the amplitude values have a single component then a threshold for the experiment is defined as the maximum amplitude value. In the case of multimodality where the amplitude values hence include multiple components then a threshold for the experiment is defined as the mean between the medians of the first two components. The method thereby obtains individual thresholds for each of the multiple ddPCR experiments.

### CROSS REFERENCE TO RELATED APPLICATION

This application is related to and claims the benefit of United Kingdom Patent Application Number GB1800551.2 filed 12 Jan. 2018, the entire contents of which are incorporated herein by reference.

### TECHNICAL FIELD

The present invention relates to a method of estimating thresholds for droplet digital polymerase chain reaction (ddPCR) experiments, and to a computer system and computer programme product for carrying out the method.

### BACKGROUND

In modern molecular science the absolute quantification of nucleic acids in a sample is often necessary or desired. Absolute quantification of DNA is important in next generation sequencing (NGS), in which the concentrations of DNA library preparations prepared for sequencing must be quantified in order to include the appropriate amount of a DNA library in a sequencing reaction, thus maximising sequencing yield. Absolute quantification of nucleic acids also enables the detection of a nucleic acid which forms only a small proportion of a sample. For instance, absolute DNA quantification can be used to detect nucleic acid sequences or mutations which are indicative of cancer within a clinical sample comprising mainly healthy tissue, or to quantify the amount of a rare allele in a sample, or to determine the number of copies of e.g. viral DNA or RNA in a given sample. Absolute quantification of DNA can also be used in conjunction with methylation-specific PCR to quantify the proportion of a given methylation pattern for a particular nucleic acid sequence within a sample.

There are at present a relatively limited number of methods of absolute quantification of a nucleic acid. These are generally based on the polymerase chain reaction (PCR), which enables amplification of DNA. RNA may be analysed by PCR if first reverse transcribed into DNA. One method of absolute DNA concentration quantification is digital PCR (dPCR), in which a PCR mixture is randomly divided into a large number of partitions. Individual PCRs are performed inside each partition and the absolute quantity of the target can be calculated based on the number of fluorescence-positive partitions. The method thus avoids the requirement for a standard curve. There are currently several commercial systems for dPCR, and with the concomitant increase in liquid biopsy analyses for cancer screening, for detection of minimal residual disease after surgery and for monitoring cancer patients, the need for high precision analyses of circulating tumour-derived nucleic acid molecules is obvious, but not necessarily implemented. Two different strategies are typically used for robust quantification in dPCR analyses. In line with standard mutation/SNP assays, primers binding to wild type sequences can be paired with probes with different fluorescent marks, one binding to the wild type version and the other to the mutated/aberrant version of the target sequence. With such a design, the ratio between mutated/aberrant and wild type DNA can be determined, and the use of an internal control would be limited to normalizing for minor technical variations, such as pipetting inaccuracies. This represents a convenient design for absolute quantification, but can be challenging for certain analyses, including DNA methylation analyses where the number of CpGs in the target region of interest often is high, and the presence of such CpG sites in the primer binding sites may bias the amplification. A commonly used alternative, is designing an assay where both primers and probe bind exclusively to the methylated version of the target. This type of analysis will require normalization using an internal control for accurate results.

One of the most commonly used platforms for digital PCR is droplet digital PCR (ddPCR), where the partitions are represented by thousands of nanolitre-scale droplets, formed by water-in-oil emulsion. ddPCR experiments generate a mixture of positive droplets, which contain the target of interest that will be further amplified, and negative droplets, which show no amplification. There is a need to quantify the number of positive droplets in order that the results of a particular experiment can be determined to be positive or negative. This is particularly critical in any type of ddPCR analysis that generate “rain”, i.e. droplets that are spread between that do not appear to be distinguishable as either positive or negative since they do not group readily with the bulk of positive or negative droplets.

### BRIEF SUMMARY

Viewed from a first aspect, the invention provides a method of estimating thresholds for droplet digital polymerase chain reaction (ddPCR) experiments, the method comprising: receiving amplitude values for multiple ddPCR experiments; for each experiment, determining if the distribution of amplitude values is multimodal or unimodal, and then: (i) in the case of unimodality where the amplitude values have a single component, defining a threshold for the experiment as the maximum amplitude value, after excluding potential outliers, and (ii) in the case of multimodality where the amplitude values hence include multiple components, defining a threshold for the experiment as the mean between the medians of the first two components; and thereby obtaining individual thresholds for each of the multiple ddPCR experiments.

With this method specific thresholds are found for each of multiple experiments that may for example be tests of multiple wells such as for quantification of DNA concentration. The thresholds may take the form of a threshold value of amplitude for determining if amplitude values should be labelled as positive or negative. The thresholds may hence allow for quantification of the number of positive droplets in each of the ddPCR experiments. By testing for unimodality first the accuracy of the method is improved and problems arising from incorrect handling of unimodal sets of amplitude values are avoided.

The method may include determining if there is unimodality in order to determine if the distribution of amplitude values is multimodal or unimodal. In some examples the step of testing if there is unimodality is done using a test such as Hartigan's dip test as implemented in the R package “diptest: Hartigan's Dip Test Statistic for Unimodality” Martin Maechler (2015). The null hypothesis of the dip test is that the distribution function is unimodal and consequently, the test alternative is non-unimodal. In some example, a multimodality is determined if the test indicates a significant p-value, i.e. lower than 5%, rejecting the null hypothesis of unimodality.

The step of testing if there is unimodality is followed by a step of identification of the components, called the “mixture components”, which make up the overall distribution of amplitude values resulting from the ddPCR experiments. The number of components and their densities may be estimated based on a bootstrap likelihood ratio test and Gaussian mixture models.

If the distribution of amplitude values is unimodal, case (i), the single component is assumed to be made of negative droplets and its density is estimated by setting the number of component to 1. If unimodality is rejected, i.e. case (ii), the number of mixture components may be determined by use of a likelihood ratio test, such as that proposed by Fraley and Raftery (C. Fraley and A. E. Raftery (2002). Model-based Clustering, Discriminant Analysis and Density Estimation Journal of the Statistical Association 97:611-631) and as implemented in the R package mclust (C. Fraley, A. E. Raftery, T. B. Murphy, and L. Scrucca (2012). mclust Version 4 for R: Normal Mixture Modeling for Model-Based Clustering, Classification, and Density Estimation Technical Report No. 597, Department of Statistics, University of Washington). This may be used to confirm that the null hypothesis of unimodality is rejected by checking if the number of components is greater than 1. If this additional check finds that there is unimodality then the method proceeds as for case (i). If not, the densities of the components are then estimated.

The method may include identification and elimination of outliers for the multimodal and/or the unimodal results. In case (i) the method may include testing for outliers and discarding outliers before the density is estimated. In case (ii) the method may include testing for outliers and discarding outliers before a final determination of the number of components and before the threshold is estimated. Identification of outliers may be done based on a comparison of the outlier value with the interquartile range (IQR) of the distribution of amplitude values. Subsequent determination of the number of positive droplets may be based on the results after the outliers are discarded.

The method may involve first processing the amplitude values for a first experiment of the multiple ddPCR experiments and then repeating the steps for second and subsequent experiments of the multiple ddPCR experiments. The output from the method may be a set of thresholds for each of the multiple experiments (for example, a set of multiple well-specific thresholds for experiments relating to multiple wells). The output may include a plot of distribution of amplitude values for each experiment, which may conveniently be marked with the estimated threshold.

The invention may extend to a method of quantifying positive droplets in ddPCR experiments by estimating thresholds for the experiments as discussed above and then determining if components of the amplitude values should be labelled as positive or negative for each of the experiments. The method may additionally or alternatively be used for determining different positive distributions. This is valuable in a multiplexing context where multiple targets need to be quantified. Thus, the invention may extend to a method of discriminating and quantifying the various positive clouds coming from individual target genes in a multiplexed experiment by estimating thresholds for the experiments as claimed in any preceding claim and then determining positive distributions of the results of the multiplexed experiment based on the estimated thresholds.

Viewed from a second aspect, the invention provides a computer programme product comprising instructions that, when executed, will configure a computer system for carrying out a method as discussed above, such as the method of the first aspect or of any optional feature thereof.

In a further aspect, the invention provides a computer system configured to carrying out a method as discussed above, such as the method of the first aspect or of any optional feature thereof. The computer system may be any computing device with appropriate programming. For example it may be a personal computer or other multi-purpose computer that receives results from ddPCR experiments carried out by another apparatus. Alternatively it may be a dedicated system for processing ddPCR results, such as a system with processors appropriately programmed to carry out the above method, with this dedicated system optionally being a part of an apparatus for automated or semi-automated ddPCR experiments.

### BRIEF DESCRIPTION OF THE DRAWING

Certain preferred embodiments of the present invention will now be described by way of example only and with reference to the accompanying drawing, in which:

FIG. 1 is a workflow for a method of threshold estimation in ddPCR experiments.

### DETAILED DESCRIPTION

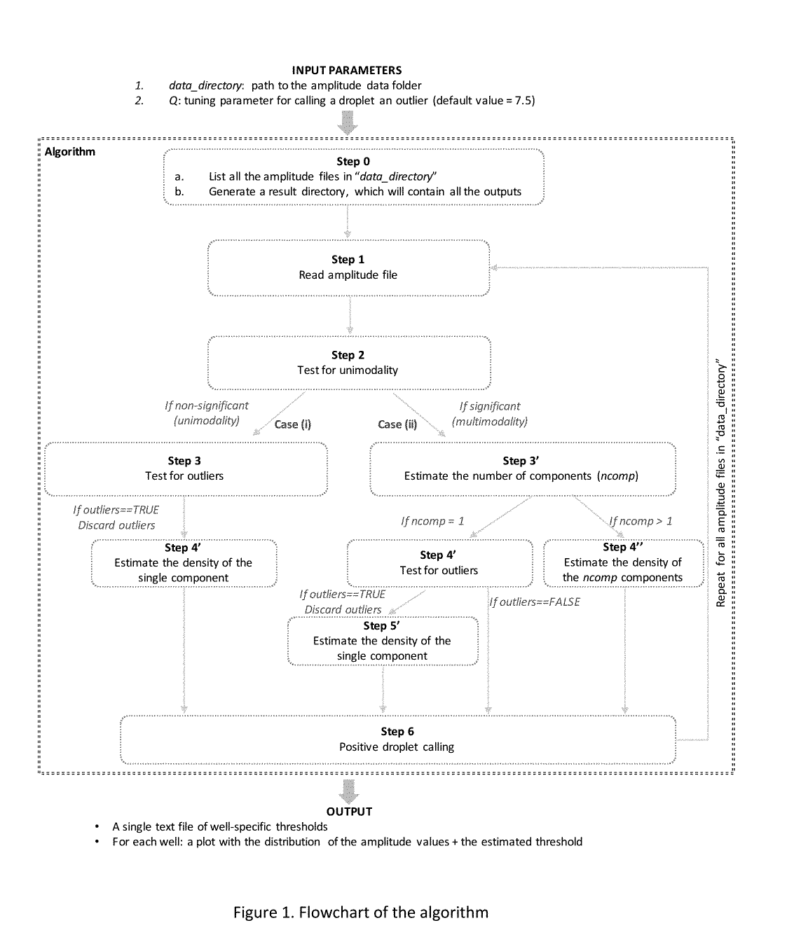

As noted above, ddPCR experiments generate a mixture of positive droplets, which contain the target of interest that will be further amplified, and negative droplets, which show no amplification. The workflow of FIG. 1 proposes a method including identifying the various components, called the “mixture components”, which make up the overall distribution of amplitude values, based on Gaussian mixture models, and then estimating thresholds for calling positive or negative droplets depending on the amplitude values. This method can conveniently be implemented using a computer algorithm. Once identified, the components are either labelled as positive signal or negative signal and a threshold is set in order to be able to quantify the number of positive droplets. The method can be extended to the identification and estimation of the various positive distributions in a multiplexing context.

As shown in the FIGURE, the main elements of the method, which may form the main modules for the related algorithm, can be implemented as below. In this example the programming language R is used for the algorithm.

a. Read Amplitude File.

This module consists in importing the amplitude data in R and extracting the target gene, the control and the well information from the amplitude file name.

b. Test for Unimodality.

Using the Hartigan's dip test, implemented in the R package diptest (Maechler 2015), the algorithm assesses whether the distribution of amplitude values is unimodal. The test is conducted at a 5% level.

c. Test for Outliers.

Outliers are identified based on the interquantile range (IQR) of the distribution of the amplitude values. The IQR is the difference between the upper and the lower quartiles (Q3 and Q1): IQR=Q3−Q1.

The cutoff for calling a droplet an outlier is defined in the algorithm as: Q3+Q*IQR, with Q being a parameter specified by the user. By default, based on empirical tests, this value was set at 7.5.

d. Estimate the Number of Mixture Components.

The number of mixture components in the distribution of amplitude values is assessed by a likelihood ratio test or LRT, based on the approach proposed by Fraley and Raftery 2002 (ibid) and implemented in the R package mclust (Fraley et al. 2012, ibid). The p-value is approximated by bootstrapping and declared significant if <0.05.

e. Estimate the Density of the Components.

A density estimate is produced for each component using a Gaussian mixture model. In case of a single component (unimodality), the number of mixture components is set to 1.

f. Positive Droplets Calling.

Depending on the number of components, two strategies are applied to estimate the threshold for calling positive droplets:
In case of a single component (unimodality), the threshold is defined as the maximum amplitude value.
In case of several components (multimodality), the threshold is defined as the mean between the medians of the two first components.

In example implementations this may be used in the context of ddPCR for DNA amplification (target amplification and control amplification). This may include partitioning of a sample into microwells or microfluidic chambers. Where the multiple ddPCR experiments will take place. The DNA-containing sample may be diluted prior to partitioning to ensure the partitions are not saturated (i.e. that not every partition contains target DNA), since the concentration of the sample cannot be quantified if the partitions are saturated. In ddPCR the partitions take the form of water droplets in a water-in-oil emulsion. Each partition may be of approximately 1 nl volume. The ddPCR may proceed in any suitable known manner, with the resulting amplitude values then being processed as discussed above.

1. A method of estimating thresholds for droplet digital polymerase chain reaction (ddPCR) experiments, the method comprising:
receiving amplitude values for multiple ddPCR experiments;
for each experiment, determining if the distribution of amplitude values is multimodal or unimodal, and then:
(i) in the case of unimodality where the amplitude values have a single component, defining a threshold for the experiment as the maximum amplitude value, and
(ii) in the case of multimodality where the amplitude values hence include multiple components, defining a threshold for the experiment as the mean between the medians of the first two components; and
thereby obtaining individual thresholds for each of the multiple ddPCR experiments.

2. A method as claimed in claim 1, comprising identifying the various components that make up the overall distribution of amplitude values in the results from the ddPCR experiments and their densities prior to a step of testing if there is unimodality in order to determine if the distribution of amplitude values is multimodal or unimodal.

3. A method as claimed in claim 1, wherein the step of determining if there is a unimodality is done using Hartigan's dip test.

4. A method as claimed in claim 1, comprising identification and elimination of outliers for the multimodal and/or the unimodal results.

5. A method as claimed in claim 4, wherein the identification of outliers may be done based on a comparison of the outlier value with an interquartile range (IQR) of the distribution of amplitude values.

6. A method as claimed in claim 1, wherein case (ii) includes the use of a mixture model to estimate the number of mixture components.

7. A method as claimed in claim 6, wherein the estimation of the number of mixture components is used to confirm unimodality by checking if the number of component's is greater than 1, and wherein if this check finds that there is unimodality then the method proceeds as for case (i).

8. A method as claimed in claim 7, wherein the number of mixture components is determined by use of a bootstrap likelihood ratio test.

9. A method as claimed in claim 1, comprising first processing the amplitude values for a first experiment of the multiple ddPCR experiments and then repeating the steps for second and subsequent experiments of the multiple ddPCR experiments.

10. A method as claimed in claim 1, wherein the output from the method is a set of thresholds for each of the multiple experiments and a plot of distribution of amplitude values for each experiment.

11. A method of quantifying positive droplets in ddPCR experiments by estimating thresholds for the experiments as claimed in claim 1 and then determining if components of the amplitude values should be labelled as positive or negative for each of the experiments based on the estimated thresholds.

12. A method of discriminating and quantifying the various positive clouds coming from individual target genes in a multiplexed experiment by estimating thresholds for the experiments as claimed in claim 1 and then determining positive distributions of the results of the multiplexed experiment based on the estimated thresholds.

13. A computer programme product comprising instructions that, when executed, will configure a computer system for carrying out a method as claimed in claim 1.
